(19) United States
(12) Patent Application Publication
(10) Pub. No.: US 2023/0420092 A1
(43) Pub. Date: Dec. 28, 2023

(54) HEALTHCARE DELIVERY ECONOMICS PREDICTION

(71) Applicant: Concord III, LLC, Seattle, WA (US)

(72) Inventor: Shawn Brockman, Seattle, WA (US)

(73) Assignee: Concord III, LLC, Seattle, WA (US)

(21) Appl. No.: 17/847,147

(22) Filed: Jun. 22, 2022

(51) Int. Cl. G16H 15/00; G06Q 30/02

(52) U.S. Cl. CPC G16H 15/00; G06Q 30/0206; G06Q 30/0201; G06Q 50/22

(57) ABSTRACT

Health care delivery economics prediction includes the receipt of a raster image of a document and the performance of optical character recognition (OCR) upon the document so as to produce parseable text. Then, a healthcare profile is created based upon a presence of a selection of words in the parseable text which had previously been associated with a particular course of treatment. Finally, a cost of the particular course of treatment is computed and stored in a database with data derived from the parseable text.

### BACKGROUND OF THE INVENTION

### Field of the Invention

The present invention relates to the technical field of patient intake in a healthcare organization and more particularly to the estimate of healthcare costs during patient intake in the healthcare organization.

### Description of the Related Art

Generally, the establishment of a customer-vendor relationship in most industries is a matter only of recording basic contact information for the customer within the records of the vendor, and the identification of a product or service sought for purchase from the vendor by the customer. There is a notion of a referral source from which the customer becomes aware of the vendor and the products or services offered for sale by the vendor, but by and large, the entire process of establishing the relationship between vendor and customer involves direct interactions between the customer and vendor without the participation of third parties.

The establishment of a healthcare provider-patient relationship differs from traditional customer-vendor in that in the healthcare context, oftentimes the patient is “referred” to the provider and the referral source is another healthcare provider. The typical circumstance is that of primary care physician to specialist physician or specialist clinic or specialist imaging center. To the extent that the individual healthcare services provided by the specialist are part of a larger healthcare picture of the patient, healthcare data must be exchanged as between the referral source—the referring healthcare provider—and the specialist. Indeed, in many instances, the healthcare information provided between two different healthcare providers in reference to a patient is more impactful than the information provided by the patient to the specialist.

In the traditional customer-vendor relationship, when establishing the relationship, the pricing of the desired product or service is known a priori or expressed to the customer at the outset of the relationship. The cost of performing the desired service or producing and distributing the desired product is known as well so that the expected profitability of the sale of the product or service to the consumer is well known. So much, however, is not the case in establishing the patient-provider relationship and, to complicate matters, the payor of the bulk of the cost of delivering healthcare services is not born by the patient but by an insurance company.

More specifically, in the latter instance, the patient oftentimes is unaware of the actual cost of delivery of the desired healthcare because it is not known in many instances, the extent of healthcare services which must be delivered to the patient depending upon an initial and possibly an ongoing diagnostic process. As well, the actual cost of delivery of the desire healthcare can be tied to the insurance carried by the patient, the amount of reimbursement subsequently proffered by the insurance carrier in response to healthcare billing after the services have already been performed, and the willingness of the provider to waive any remaining difference between the billed cost of service and the amount reimbursed by the insurance carrier. Thus, predicting the economics of healthcare services at the time of establishing a patient-provider relationship can be challenging.

### BRIEF SUMMARY OF THE INVENTION

Embodiments of the present invention address technical deficiencies of the art in respect to the automated management of patient intake in a healthcare organization. To that end, embodiments of the present invention provide for a novel and non-obvious method for health care delivery economics prediction. Embodiments of the present invention also provide for a novel and non-obvious computing device adapted to perform the foregoing method. Finally, embodiments of the present invention provide for a novel and non-obvious data processing system incorporating the foregoing device in order to perform the foregoing method.

In one embodiment of the invention, a health care delivery economics prediction method includes receiving a raster image of a document and performing OCR upon the document to produce parseable text. Then, a healthcare profile can be created based upon a presence of a selection of words in the parseable text previously associated with a particular course of treatment. Finally, a cost of the particular course of treatment can be computed and the cost can be stored in a database with data derived from the parseable text. Optionally, a margin of profitability also can be computed for the course of treatment based upon the computed cost and then the margin can be stored with the cost in the database. As another option, a report of the course of treatment and computed cost can be transmitted to a patient listed in the parseable text.

As a further option, the computed costs can be aggregated for multiple different received raster documents of like healthcare profile. Then, an actual cost of delivery of the course of treatment can be received for corresponding patients associated with the documents. Statistics then can be stored in a data store, the statistics having been determined from the actual cost of delivery for the corresponding patients. These statistics subsequently can be used in computing the cost of the particular cost of treatment for a newly received raster image.

In another embodiment of the invention, a data processing system is adapted for health care delivery economics prediction. The system includes a host computing platform that has one or more computers, each with memory and one or processing units including one or more processing cores. The system also includes a health care delivery economics prediction module. The module includes computer program instructions enabled while executing in the memory of at least one of the processing units of the host computing platform to receive a raster image of a document and performing OCR upon the document to produce parseable text, to generate a healthcare profile based upon a presence of a selection of words in the parseable text previously associated with a particular course of treatment, to compute a cost of the particular course of treatment, and to store the cost in a database with data derived from the parseable text.

In this way, the technical deficiencies of the prediction of the costs of delivering healthcare services to a patient are overcome owing to a prior determination of the services required as determined from the content of an inbound facsimile document proposing the patient-provider relationship, and the historical knowledge of the cost of delivering those determined services. Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration reflecting different aspects of a process of [statement of the invention];

FIG. 2 is a block diagram depicting a data processing system adapted to perform one of the aspects of the process of FIG. 1; and,

FIG. 3 is a flow chart illustrating one of the aspects of the process of FIG. 1.

### DETAILED DESCRIPTION OF THE INVENTION

Embodiments of the invention provide for health care delivery economics prediction. In accordance with an embodiment of the invention, an OCR process performs OCR upon a facsimile image and extracts therefrom demographic information regarding a prospective patient and at least one term pertaining to a healthcare diagnosis. A treatment profile is then constructed in reference to the demographic information and term pertaining to the healthcare diagnosis. Once the treatment profile has been constructed, a base cost for the treatment profile is retrieved from a table and, optional, the base cost is then modified according to additional terms extracted from the facsimile image during OCR. The modified cost is then included in a printed report to the patient and the modified cost is stored in a datastore of predicted costs for delivering healthcare services respect to the healthcare diagnosis. In this way, the predicted costs can be retrieved for subsequently received facsimile images pertaining to the healthcare diagnosis.

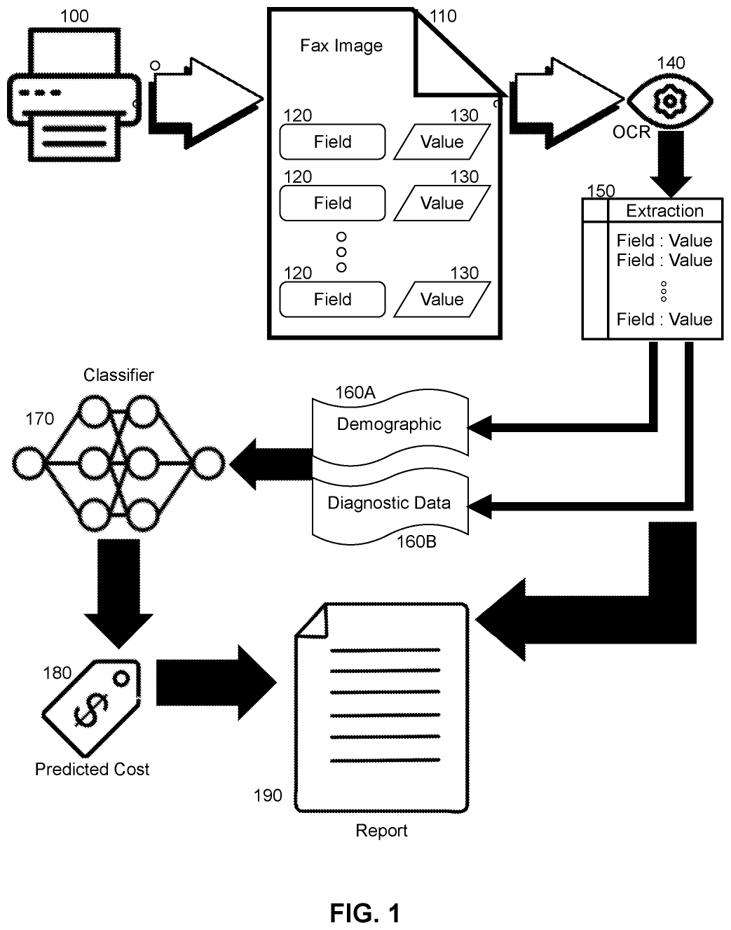

In illustration of one aspect of the embodiment, FIG. 1 pictorially shows a process of health care delivery economics prediction. As shown in FIG. 1, A raster image 110 of a facsimile received from facsimile transmission device 100 and the fields 120 and respective values 130 are transformed through OCR 140 to an extraction set 150 of field-value pairs. From the extraction 150, ones of the field-value pairs associated with demographic information 160A such as gender, age and gender can be selected, along with ones of the field-value pairs associated with diagnostic data 160B, such as terms associated with a particular symptom, treatment or disease. The demographic information 160A and the diagnostic information 160B are then submitted to a classifier 170 adapted to predict a treatment cost for a patient of the demographic information 160 for the disease associated with the diagnostic information 160B.

Of note, the classifier 170 can produce the predicted cost 180 based upon the parallel submission of the demographic information 160A and the diagnostic information 160B, or the classifier 170 can be chained structures wherein a first structure predicts a base cost for the diagnostic information 160B which base cost is then provided to a second structure with the demographic information 160B in order to provide a modified form of the base cost. In either instance, the classifier 170 then incorporated the predicted cost 180 into a report 190 aggregating the demographic information 160A and the diagnostic information 160B with the predicted cost 180.

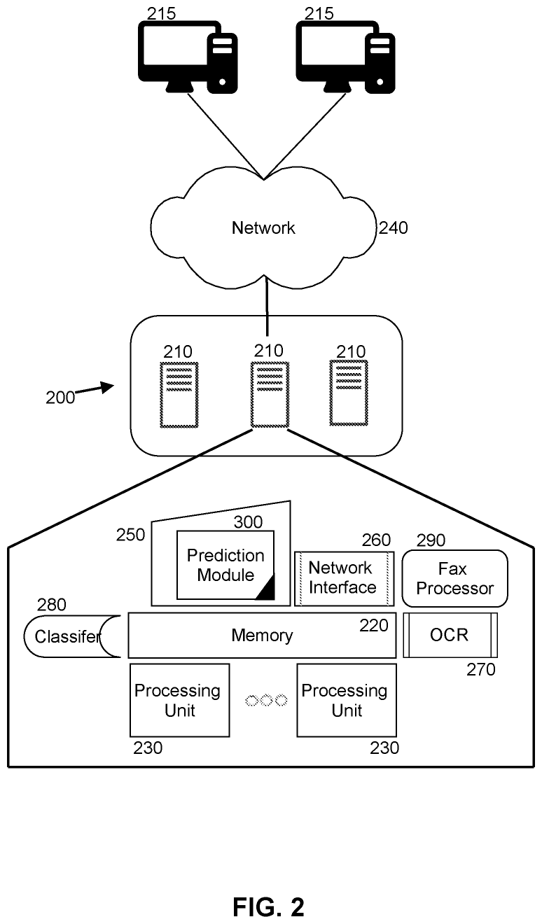

Aspects of the process described in connection with FIG. 1 can be implemented within a data processing system. In further illustration, FIG. 2 schematically shows a data processing system adapted to perform health care delivery economics prediction. In the data processing system illustrated in FIG. 1, a host computing platform 200 is provided. The host computing platform 200 includes one or more computers 210, each with memory 220 and one or more processing units 230. At least one of the computers 210 includes a fax processor 290 adapted to receive a fax signal in order to persist a raster image of a document. At least one of the computers 210 also includes an OCR module 270 configured to perform OCR on the raster image of the document in order to produce computer readable text.

Importantly, a classifier 280 can be stored in the memory 220 of at least one of the computers 210. The classifier 280 is a data structure adapted to receive as input data pertaining to diagnostic information such as a symptom, disease or treatment and to respond with a cost prediction of the treatment based upon prior machine learned correlation between the diagnostic information and the cost prediction. To that end, the classifier 280 can range from a simplistic table associated input keywords without cost values to a deep neural network trained with diagnostic inputs annotated with known costs. Notably, whether a simple table or complex deep neural network, the classifier 280 is adapted to modify its correlations based upon the contemporaneously submitted ground truths of actual cost of treatment for particular input diagnostic data. As well, optionally, the classifier 280 can receive as input data demographic information in compliment to the diagnostic information in generating the cost prediction.

As can be seen, the computers 210 of the host computing platform (only a single computer shown for the purpose of illustrative simplicity) can be co-located within one another and in communication with one another over a local area network, or over a data communications bus, or the computers can be remotely disposed from one another and in communication with one another through network interface 260 over a data communications network 240 and also in communicative coupling to different remotely disposed client devices 215. Notably, a computing device 250 including a non-transitory computer readable storage medium can be included with the data processing system 200 and accessed by the processing units 230 of one or more of the computers 210. The computing device stores 250 thereon or retains therein a prediction program module 300 that includes computer program instructions which when executed by one or more of the processing units 230, performs a programmatically executable process for health care delivery economics prediction.

Specifically, the program instructions during execution receive from the fax processor 290 a raster image of a document and provide the raster image to OCR 270 module which in turn produces an index of field-value pairs representative of form based fields and corresponding values for the fields. The program instructions then, in reference to a list of known fields, selects one or more different fields in the index associated with diagnostic information and submits the corresponding values to the classifier 280. The program instructions subsequently receive from the classifier 280 a predicted cost which the program instructions then incorporate into a report in connection with diagnostic information and demographic information additionally selected in the index.

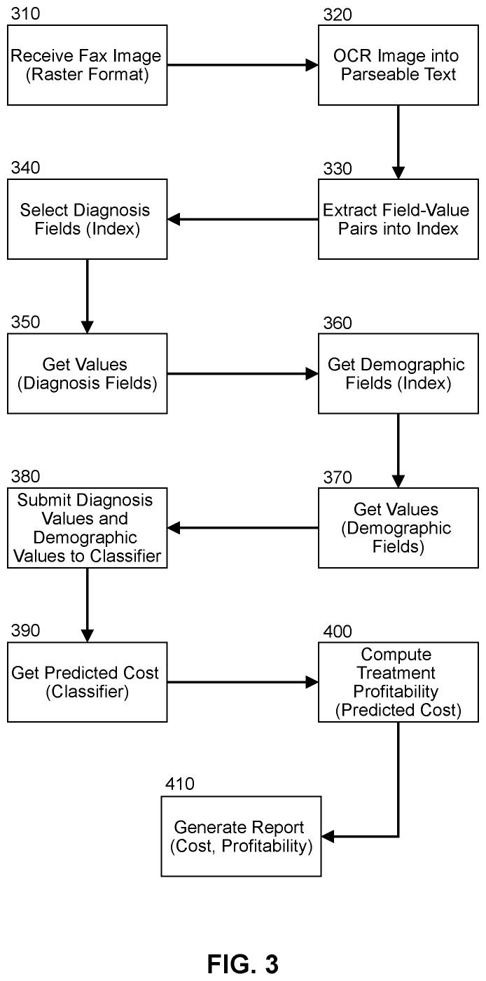

In further illustration of an exemplary operation of the module, FIG. 3 is a flow chart illustrating one of the aspects of the process of FIG. 1. Beginning in block 310, a raster image of a facsimile document is received and in block 320 the raster image is subjected to OCR in order to produce parseable text. In block 330, field-value pairs are extracted from the parseable text into an index and in block 340, one or more different diagnosis fields of the index are selected. As such, in block 350 corresponding values for the selected fields are retrieved. Similarly, in block 360 one or more or different demographic fields of the index are selected and in block 370 corresponding values for the selected fields are retrieved. In block 380, the diagnosis values and the demographic values are submitted to a classifier. Subsequently, in block 390, a predicted cost is received from the classifier and in block 400 a treatment profitability is computed based upon the difference between the predicted cost of treatment and the known reimburseable fees afforded for the treatment. Finally, a report is generated incorporating the predicted cost and the computed profitability.

Of import, the foregoing flowchart and block diagram referred to herein illustrate the architecture, functionality, and operation of possible implementations of systems, methods, and computing devices according to various embodiments of the present invention. In this regard, each block in the flowchart or block diagrams may represent a module, segment, or portion of instructions, which includes one or more executable instructions for implementing the specified logical function or functions. In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

More specifically, the present invention may be embodied as a programmatically executable process. As well, the present invention may be embodied within a computing device upon which programmatic instructions are stored and from which the programmatic instructions are enabled to be loaded into memory of a data processing system and executed therefrom in order to perform the foregoing programmatically executable process. Even further, the present invention may be embodied within a data processing system adapted to load the programmatic instructions from a computing device and to then execute the programmatic instructions in order to perform the foregoing programmatically executable process.

To that end, the computing device is a non-transitory computer readable storage medium or media retaining therein or storing thereon computer readable program instructions. These instructions, when executed from memory by one or more processing units of a data processing system, cause the processing units to perform different programmatic processes exemplary of different aspects of the programmatically executable process. In this regard, the processing units each include an instruction execution device such as a central processing unit or “CPU” of a computer. One or more computers may be included within the data processing system. Of note, while the CPU can be a single core CPU, it will be understood that multiple CPU cores can operate within the CPU and in either instance, the instructions are directly loaded from memory into one or more of the cores of one or more of the CPUs for execution.

Aside from the direct loading of the instructions from memory for execution by one or more cores of a CPU or multiple CPUs, the computer readable program instructions described herein alternatively can be retrieved from over a computer communications network into the memory of a computer of the data processing system for execution therein. As well, only a portion of the program instructions may be retrieved into the memory from over the computer communications network, while other portions may be loaded from persistent storage of the computer. Even further, only a portion of the program instructions may execute by one or more processing cores of one or more CPUs of one of the computers of the data processing system, while other portions may cooperatively execute within a different computer of the data processing system that is either co-located with the computer or positioned remotely from the computer over the computer communications network with results of the computing by both computers shared therebetween.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A health care delivery economics prediction method comprising:
receiving a raster image of a document and performing OCR upon the document to produce parseable text;
generating a healthcare profile based upon a presence of a selection of words in the parseable text previously associated with a particular course of treatment;
computing a cost of the particular course of treatment; and,
storing the cost in a database with data derived from the parseable text.

2. The method of claim 1, further comprising computing a margin of profitability for the course of treatment and storing the margin with the cost in the database.

3. The method of claim 1, further comprising transmitting a report of the course of treatment and computed cost to a patient listed in the parseable text.

4. The method of claim 1, further comprising aggregating computed costs for multiple different received raster documents of like healthcare profile, receiving an actual cost of delivery of the course of treatment for corresponding patients associated with the documents and storing statistics determined from the actual cost of delivery for the corresponding patients in a data store for use in computing the cost of the particular cost of treatment for a newly received raster image.

5. A data processing system adapted for health care delivery economics prediction, the system comprising:
a host computing platform comprising one or more computers, each with memory and one or processing units including one or more processing cores; and,
a health care delivery economics prediction module comprising computer program instructions enabled while executing in the memory of at least one of the processing units of the host computing platform to perform:
receiving a raster image of a document and performing optical character recognition (OCR) upon the document to produce parseable text;
generating a healthcare profile based upon a presence of a selection of words in the parseable text previously associated with a particular course of treatment;
computing a cost of the particular course of treatment; and,
storing the cost in a database with data derived from the parseable text.

6. The system of claim 5, wherein the program instructions further compute a margin of profitability for the course of treatment and storing the margin with the cost in the database.

7. The system of claim 5, wherein the program instructions further transmit a report of the course of treatment and computed cost to a patient listed in the parseable text.

8. The system of claim 5, wherein the program instructions further aggregate computed costs for multiple different received raster documents of like healthcare profile, receive an actual cost of delivery of the course of treatment for corresponding patients associated with the documents and store statistics determined from the actual cost of delivery for the corresponding patients in a data store for use in computing the cost of the particular cost of treatment for a newly received raster image.

9. A computing device comprising a non-transitory computer readable storage medium having program instructions stored therein, the instructions being executable by at least one processing core of a processing unit to cause the processing unit to perform a method for health care delivery economics prediction, the method including:
receiving a raster image of a document and performing OCR upon the document to produce parseable text;
generating a healthcare profile based upon a presence of a selection of words in the parseable text previously associated with a particular course of treatment;
computing a cost of the particular course of treatment; and,
storing the cost in a database with data derived from the parseable text.

10. The device of claim 9, wherein the processing unit further performs computing a margin of profitability for the course of treatment and storing the margin with the cost in the database.

11. The device of claim 9, wherein the processing unit further performs transmitting a report of the course of treatment and computed cost to a patient listed in the parseable text.

12. The device of claim 9, wherein the processing unit further performs aggregating computed costs for multiple different received raster documents of like healthcare profile, receiving an actual cost of delivery of the course of treatment for corresponding patients associated with the documents and storing statistics determined from the actual cost of delivery for the corresponding patients in a data store for use in computing the cost of the particular cost of treatment for a newly received raster image.
